(19) United States
(12) Patent Application Publication
(10) Pub. No.: US 2003/0109906 A1
(43) Pub. Date: Jun. 12, 2003

(54) Low level light therapy for the treatment of stroke

(76) Inventor: Jackson Streeter, Reno, NV (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/287,432

(22) Filed: Nov. 1, 2002

Related U.S. Application Data
(60) Provisional application No. 60/336,436, filed on Nov. 1, 2001.
(60) Provisional application No. 60/369,260, filed on Apr. 2, 2002.

(51) Int. Cl. A61N 5/06

(52) U.S. Cl. 607/88; 607/89

(57) ABSTRACT

Therapeutic methods for the treatment of stroke are described, the methods including delivering a neuroprotective effective amount of light energy having a wavelength in the visible to near-infrared wavelength range to that area of the brain containing the area of primary infarct. The neuroprotective effective amount of light energy is a predetermined power density (mW/cm²) at the level of the brain tissue being treated, and is delivered by determining a surface power density of the light energy that is sufficient to deliver the predetermined power density of light energy to the brain tissue.

### RELATED APPLICATION INFORMATION

This application claims priority under 35 U.S.C. § 119(e) to U.S. Provisional Application Ser. No. 60/336,436 filed Nov. 1, 2001 and U.S. Provisional Application Serial No. 60/369,260, filed Apr. 2, 2002.

### FIELD OF THE INVENTION

The present invention relates in general to therapeutic methods for the treatment of stroke, and more particularly to novel methods for treating stroke using light therapy.

### BACKGROUND OF THE INVENTION

Stroke, also called cerebrovascular accident (CVA), is a sudden disruption of blood flow to a discrete area of the brain that is brought on by a clot lodging in an artery supplying that area of that brain, or cerebral hemorrhage due to a ruptured aneurysm or a burst artery. The consequence of stroke is a loss of function in the affected brain region and concomitant loss of bodily function in areas of the body controlled by the affected brain region. Depending upon the extent and location of the primary insult in the brain, loss of function varies greatly from mild or severe, and may be temporary or permanent. Lifestyle factors such as smoking, diet, level of physical activity and high cholesterol increase the risk of stroke, and thus stroke is a major cause of human suffering in developed nations. Stroke is the third leading cause of death in most developed nations, including the United States.

Until recently, stroke treatment was restricted to providing basic life support at the time of the stroke, followed by rehabilitation. Recently, new drug therapies have taken the approach of breaking up blood clots or protecting surviving by at-risk neurons from further damage.

Thrombolytic therapy includes aspirin or intravenous heparin to prevent further clot formation and to maintain blood flow after an ischemic stroke. Thrombolytic drugs include tissue plasminogen activator (TPA) and genetically engineered version thereof, and streptokinase. However, streptokinase does not appear to improve outlook even when administered early (within three hours of stroke). TPA when administered early appears to substantially improve prognosis, but slightly increases the risk of death from hemorrhage. In addition, over half of stroke patients arrive at the hospital more than three hours after a stroke, and even if they arrive quickly a CT scan must first confirm that the stroke is not hemorrhagic, which delays administration of the drug. Also, patients taking aspirin or other blood thinners, and patients with clotting abnormalities should not be given TPA.

Neuroprotective drugs have been described that target surviving but endangered neurons in a zone of risk surrounding the area of primary infarct. Such drugs are aimed at slowing down or preventing the death of such neurons, to reduce the extent of brain damage. Certain neuroprotective drugs are anti-excitotoxic, i.e., work to block the excitotoxic effects of excitatory amino acids such as glutamate that cause cell membrane damage under certain conditions. Other drugs such as citicoline works by repairing damaged cell membranes. Lazaroids such as Tirilazed (Freedox) counteract oxidative stress produced by oxygen-free radicals produced during stroke. Other drugs for stroke treatment include agents that block the enzyme known as PARP, and calcium-channel blockers such as nimodipine (Nimotop) that relax the blood vessels to prevent vascular spasms that further limit blood supply. However, the effect of nimodipine is reduced if administered beyond six hours after a stroke and it is not useful for ischemic stroke. In addition, drug therapy includes the risk of adverse side effects and immune responses.

Surgical treatment for stroke includes carotid endarterectomy, which appears to be especially effective for reducing the risk of stroke recurrence for patients exhibiting arterial narrowing of more than 70%. However, endarterectomy is highly invasive, and risk of stroke recurrence increases temporarily after surgery. Experimental stroke therapies include an angiography-type or angioplasty-type procedure using a thin catheter to remove or reduce the blockage from a clot. However, such procedures have extremely limited availability and increase the risk of embolic stroke. Other surgical interventions, such as those to repair an aneurysm before rupture remain controversial because of disagreement over the relative risks of surgery and leaving the aneurysm untreated.

High energy laser radiation is now well accepted as a surgical tool for cutting, cauterizing, and ablating biological tissue. High energy lasers are now routinely used for vaporizing superficial skin lesions and, to make deep cuts. For a laser to be suitable for use as a surgical laser, it must provide laser energy at a power sufficient to heart tissue to temperatures over 50° C. Power outputs for surgical lasers vary from 1-5 W for vaporizing superficial tissue, to about 100 W for deep cutting.

In contrast, low level laser therapy involves therapeutic administration of laser energy to a patient at vastly lower power outputs than those used in high energy laser applications, resulting in desirable biostimulatory effects while leaving tissue undamaged. For example, in rat models of myocardial infarction and ischemia-reperfusion injury, low energy laser irradiation reduces infarct size and left ventricular dilation, and enhances angiogenesis in the myocardium. (Yaakobi et al., J. Appl. Physiol. 90, 2411-19 (2001)). Low level laser therapy has been described for treating pain, including headache and muscle pain, and inflammation. However, low level laser therapy for the treatment of stroke has not been described.

In addition, known low level laser therapy methods are circumscribed by setting selected parameters within specified limits. For example, known methods include setting the power output of the laser source at very low levels of 5 mW to 70 mW, low dosages at about 1-10 Joule/cm², and time periods of application of the laser energy at twenty seconds to minutes. However, other parameters can be varied in the use of low level laser therapy. In particular, known low level laser therapy methods have not accounted for other factors that contribute to the photon density that actually is delivered to the tissue and may play key roles in the efficacy of low level laser therapy.

Against this background, a high level of interest remains in finding new and improve therapeutic methods for the treatment of stroke. In particular, a need remains for relatively inexpensive and non-invasive approaches to treating stroke that also avoid the limitations of drug therapy.

### SUMMARY OF THE INVENTION

The low level light therapy methods for the treatment of stroke is based in part on the new and surprising discovery that power density (i.e., power per unit area) of the light energy applied to tissue appears to be a very important factor in determining the relative efficacy of low level light therapy, and particularly with respect to treating and saving surviving but endangered neurons in a zone of danger surrounding the primary infarct after a stroke or cerebrovascular accident (CVA).

In accordance with one embodiment there are provided methods directed toward the treatment of stroke in a subject in need of such treatment. The methods include delivering a neuroprotective effective amount of a light energy having a wavelength in the visible to near-infrared wavelength range to a target area of the brain of the subject that includes an infarct, wherein delivering the neuroprotective effective amount of light energy includes delivering a predetermined power density of light energy through the skull to the target area of the brain.

In one embodiment the predetermined power density is a power density of at least about 0.01 mW/cm². The predetermined power density is typically selected from the range of about 0.01 mW/cm² to about 100 mW/cm², including from about 0.01 mW/cm² to about 15 mW/cm² and from about 2 mW/cm² to about 50 mW/cm².

In preferred embodiments, the methods encompass using light energy having a wavelength of about 630 nm to about 904 nm, and in one embodiment the light energy has a wavelength of about 780 nm to about 840 nm. The light energy is preferably from a coherent source (i.e. a laser), but light from non-coherent sources may also be used.

In preferred embodiments, the methods encompass placing a light source in contact with a region of skin that is either adjacent the area of the brain that includes the area of infarct, contralateral to such area, or a combination of the foregoing, and then administering the neuroprotective effective amount of light energy to the area of the brain by delivering the predetermined power density. In addition, to deliver the predetermined power density to the area of the brain, the methods encompass determining a surface power density of the light energy sufficient for the light energy to penetrate the skull. The determination of the required surface power density, which is relatively higher than the predetermined power density to be delivered to the brain area being treated, takes into account factors that attenuate power density as it travels through tissue, including skin pigmentation, and location of the brain area being treated, particularly the distance of the brain area from the skin surface where the light energy is applied.

In accordance with another embodiment, there is provided a method of increasing the production of ATP by neurons. The method comprises irradiating neurons with light energy having a wavelength in the near infrared to visible portion of the electromagnetic spectrum for at least about 1 second, where the power density of said light energy at the neurons is at least about 0.01 mW/cm².

### BRIEF DESCRIPTION OF THE DRAWINGS

FIG. 1 is a perspective view of a first embodiment of a light therapy device; and

FIG. 2 is a block diagram of a control circuit for the light therapy device, according to one embodiment of the invention.

### DETAILED DESCRIPTION OF THE PREFERRED EMBODIMENTS

The low level light therapy methods for the treatment of stroke described herein are practiced and described using, for example, a low level laser therapy apparatus such as that shown and described in U.S. Pat. Nos. 6,214,035, 6,267,780, 6,273,905 and 6, 290,714, which are all herein incorporated by reference together with references contained therein.

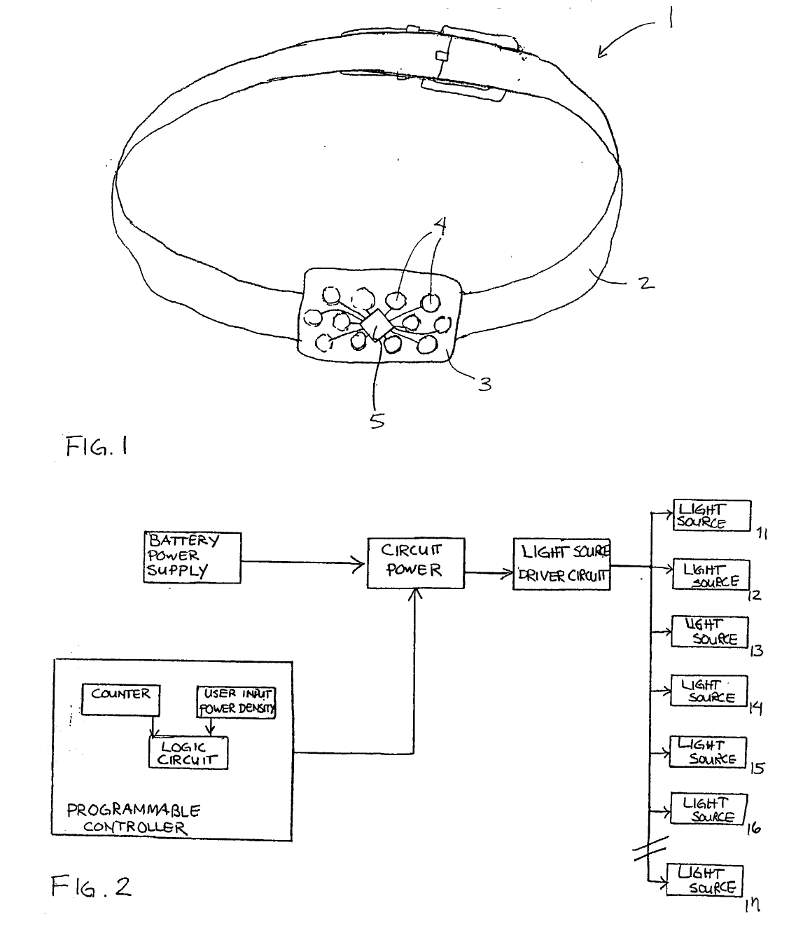

Another suitable light therapy apparatus is that illustrated in FIG. 1. The illustrated device 1 includes a flexible strap 2 with a securing means, the strap adapted for securing the device over an area of the subject's body, one or more light energy sources 4 disposed on the strap 2 or on a plate or enlarged portion of the strap 3, capable of emitting light energy having a wavelength in the visible to near-infrared wavelength range, a power supply operatively coupled to the light source or sources, and a programmable controller 5 operatively coupled to the light source or sources and to the power supply. Based on the surprising discovery that control or selection of power density of light energy is an important factor in determining the efficacy of light energy therapy, the programmable controller is configured to select a predetermined surface power density of the light energy sufficient to deliver a predetermined subsurface power density to a body tissue to be treated beneath the skin surface of the area of the subject's body over which the device is secured.

The light energy source or sources are capable of emitting the light energy at a power sufficient to achieve the predetermined subsurface power density selected by the programmable controller. It is presently believed that tissue will be most effectively treated using subsurface power densities of light of at least about 0.01 mW/cm² and up to about 100 mW/cm², including about 0.05, 0.1, 0.5, 1, 5, 10, 15, 20, 30, 40, 50, 60, 70, 80, and 90 mW/cm². In one embodiment, power densities of about 20 mW/cm² to about 50 mW/cm² are used. To attain subsurface power densities within these stated ranges, taking into account attenuation of the energy as it travels through bone, body tissue, and fluids from the surface to the target tissue, surface power densities of from about 100 mW/cm² to about 500 mW/cm² will typically be required, but also possibly to a maximum of about 1000 mW/cm² To achieve such surface power densities, preferred light energy sources, or light energy sources in combination, are capable of emitting light energy having a total power output of at least about 25 mW to about 500 mW, including about 30, 50, 75, 100, 150, 200, 250, 300, and 400 mW, but may also be up to a maximum of about 1000 mW. It is believed that the subsurface power densities of at least about 0.01 mW/cm² and up to about 100 mW/cm² in terms of the power density of energy that reaches the subsurface tissue are especially effective at producing the desired biostimulative effects on tissue being treated.

The strap is preferably fabricated from an elastomeric material to which is secured any suitable securing means, such as mating Velcro strips, snaps, hooks, buttons, ties, or the like. Alternatively, the strap is a loop of elastomeric material sized appropriately to fit snugly over a particular body part, such as a particular arm or leg joint, or around the chest or head. The precise configuration of the strap is subject only to the limitation that the strap is capable of maintaining the light energy sources in a select position relative to the particular area of the body or tissue being treated. In an alternative embodiment, a strap is not used and instead the light source or sources are incorporated into or attachable onto a light cap which fits securely over the head thereby holding the light source or sources in proximity to the patient's head for treatment. The light cap is preferably constructed of a stretchable fabric or mesh comprising materials such as Lycra or nylon. The light source or sources are preferably removably attached to the cap so that they may be placed in the position needed for treatment of a stroke or CVA in any portion of the brain.

In the exemplary embodiment illustrated in FIG. 1, a light therapy device includes a flexible strap and securing means such as mating Velcro strips configured to secure the device around the head of the subject. The light source or sources are disposed on the strap, and in one embodiment are enclosed in a housing secured to the strap. Alternatively, the light source or sources are embedded in a layer of flexible plastic or fabric that is secured to the strap. In any case, the light sources are secured to the strap so that when the strap is positioned around a body part of the patient, the light sources are positioned so that light energy emitted by the light sources is directed toward the skin surface over which the device is secured. Various strap configurations and spatial distributions of the light energy sources are contemplated so that the device can be adapted to treat different tissues in different areas of the body.

FIG. 2 is a block diagram of a control circuit according to one embodiment of the light therapy device. The programmable controller is configured to select a predetermined surface power density of the light energy sufficient to deliver a predetermined subsurface power density, preferably about 0.01 mW/cm² to about 100 mW/cm², including about 0.01 mW/cm² to about 15 mW/cm² and about 20 mW/cm² to about 50 mW/cm² to the infarcted area of the brain. The actual total power output if the light energy sources is variable using the programmable controller so that the power of the light energy emitted can be adjusted in accordance with required surface power energy calculations as described below.

Particularly suitable for the methods of treating stroke is a low level light apparatus including a handheld probe for delivering the light energy. The probe includes a light source of light energy having a wavelength in the visible to near-infrared wavelength range, i.e., from about 630 to about 904 nm, preferably about 780 nm to about 840 nm, including about 790, 800, 810, 820, and 830 nm. Preferred probes include, for example, a single source or laser diode that provides about 25 mW to about 500 mW of total power output, and multiple sources or laser diodes that together are capable of providing at least about 25 mW to about 500 mW of total power output. Probes and sources having power capacities outside of these limits may also be used in the methods according to preferred embodiments. The actual power output is variable using a control unit electronically coupled to the probe, so that power of the light energy emitted can be adjusted in accordance with required power density calculations as described below. In one embodiment, the diodes used are continuously emitting GaAIAs laser diodes having a wavelength of about 830 nm. In another embodiment, a laser source is used having a wavelength of about 808 nm. It has also been found that an intermediate wavelength of about 739 nm appears to be suitable for penetrating the skull, although other wavelengths are also suitable and may also be used.

Preferred methods are based at least in part on the finding that given a select wave of light energy it is the power density of the light energy (i.e., light intensity or power per unit area, in W/cm²) delivered to tissue, and not the power of the light source used nor the dosage of the energy used per se, that appears to be an important factor in determining the relative efficacy of low level light therapy. In the methods described herein, power density as delivered to a brain area including the area of infarct after a stroke appears to be an important factor in using low level light therapy to treat and save surviving but endangered neurons in a zone of danger surrounding the infarcted area. Without being bound by theory, it is believed that only light energy delivered within a certain range of power densities provides the required biostimulative effect on the intracellular environment, such that proper function is returned to previously nonfunctioning or poorly functioning mitochondria in at-risk neurons.

The term “neurodegeneration” refers to the process of cell destruction resulting from primary destructive events such as stroke or CVA, and also secondary, delayed and progressive destructive mechanisms that are invoked by cells due to the occurrence of the primary destructive event. Primary destructive events include disease processes or physical injury or insult, including stroke, but also include other diseases and conditions such as multiple sclerosis, amylotrophic lateral sclerosis, epilepsy, Alzheimer's disease, dementia resulting from other causes such as AIDS, cerebral ischemia including focal cerebral ischemia, and physical trauma such as crush or compression injury in the CNS, including a crush or compression injury of the brain, spinal cord, nerves or retina, or any acute injury or insult producing neurodegeneration. Secondary destructive mechanisms include any mechanism that leads to the generation and release of neurotoxic molecules, including apoptosis, depletion of cellular energy stores because of changes in mitochondrial membrane permeability, release or failure in the reuptake of excessive glutamate, reperfusion injury, and activity of cytokines and inflammation. Both primary and secondary mechanisms contribute to forming a “zone of danger” for neurons, wherein the neurons in the zone have at least temporarily survived the primary destructive event, but are at risk of dying due to processes having delayed effect.

The term “neuroprotection” refers to a therapeutic strategy for slowing or preventing the otherwise irreversible loss of neurons due to neurodegeneration after a primary destructive event, whether the neurodegeneration loss is due to disease mechanisms associated with the primary destructive event or secondary destructive mechanisms.

The term “neuroprotective effective” as used herein refers to a characteristic of an amount of light energy, wherein the amount is a power density of the light energy measured in mW/cm ². The amount of light energy achieves the goal of preventing, avoiding, reducing or eliminating neurodegeneration.

Thus, a method for the treatment of stroke in a subject in need of such treatment involves delivering a neuroprotective effective amount of light energy having a wavelength in the visible to near-infrared wavelength range to a target area of the brain of the subject that includes the area of infarct, i.e. to neurons within the “zone of danger.” Delivering the neuroprotective amount of light energy includes selecting a surface power density of the light energy sufficient to deliver the predetermined power density of light energy to the target area of the brain. The predetermined power density to be delivered to the tissue is selected to be at least about 0.01 mW/cm². In one embodiment, the predetermined power density is selected from the range of about 0.01 mW/cm² to about 100 mW/cm² To deliver the predetermined power density at the level of the brain tissue, a required, relatively greater surface power density of the light energy is calculated taking into account attenuation of the light energy as it travels from the skin surface through various tissues including skin, bone and brain tissue. Factors known to affect penetration and to be taken into account in the calculation include skin pigmentation, the presence and color of hair over the area to be treated, and the location of the affected brain region, particularly the depth of the area to be treated relative to the surface. For example, to obtain a desired power density of 50 mW/cm² in the brain at a depth of 3 cm below the surface may require a surface power density of 500 mW/cm². The higher the level of skin pigmentation, the higher the required surface power density to deliver a predetermined power density of light energy to a subsurface brain site.

The wavelength of the light energy is selected from the range of about 630 nm to about 904 nm, and of course is dependent on the source of light energy used one embodiment, using light apparatus including GaAIAs laser diodes, the light energy has a wavelength of about 830 mn.

In preferred embodiments, the light source used in light therapy is a coherent source (i.e. a laser), and/or the light is substantially monochromatic (i.e. one wavelength or a very narrow band of wavelengths).

To treat a patient suffering from the effects of stroke, the light source is placed in contact with a region of skin, for example on the scalp, adjacent the area of the affected area of the brain that has been identified such as by using standard medical imaging techniques. Then a surface power density calculation is performed which takes into account factors including skull thickness of the patient, skin coloration, distance to affected site within the brain, etc. that affect penetration and thus power density at the affected site. The power and other parameters are then adjusted according to the results of the calculation.

The precise power density selected for treating the patient depends on a number of factors, including the specific wavelength of light selected, the type of CVA (ischemic or hemorrhagic), the clinical condition of the subject including the extent of brain area affected, and the like. Similarly, it should be understood that the power density of light energy to be delivered to the affected brain area may be adjusted to be combined with any other therapeutic agent or agents, especially pharmaceutical neuroprotective agents to achieve the desired biological effect. The selected power density will again depend on a number of factors, including the specific light energy wavelength chosen, the individual additional therapeutic agent or agents chosen, and the clinical condition of the subject.

In preferred embodiments, the treatment proceeds continuously for a period of about 30 seconds to about 2 hours, more preferably for a period of about 1 to 20 minutes. The treatment may be terminated after one treatment period, or the treatment may be repeated with preferably about 1 to 2 days passing between treatments. The length of treatment time and frequency of treatment periods depends on several factors, including the functional recovery of the patient and the results of imaging analysis of the infarct.

During the treatment, the light energy may be continuously provided, or it may be pulsed. If the light is pulsed, the pulses are preferably at least about 10 ns long and occur at a frequency of up to about 100 Hz. Continuous wave light may also be used.

It has been discovered that treatment of stroke using low level light therapy is more effective if treatment begins several hours after the stroke has occurred. This is a surprising result, in that the thrombolytic therapies currently in use for treatment of stroke must begin within a few hours of the stroke. Because oftentimes many hours pass before a person who has suffered a stroke receives medical treatment, the short time limit for initiating thrombolytic therapy excludes many patients from treatment. Consequently, the present methods may be used to treat a greater percentage of stroke patients.

Although not wanting to be bound by theory, it is believed that the benefit in delaying treatment occurs because of the time needed for induction of ATP production, and/or the possible induction of angiogenesis in the region surrounding the infarct. Thus, in accordance with one preferred embodiment, the light therapy for the treatment of stroke occurs preferably about 6 to 24 hours after the onset of stroke symptoms, more preferably about 12 to 24 hours after the onset of symptoms. It is believed, however, that if treatment begins after about 2 days, its effectiveness will be greatly reduced.

### EXAMPLE

An in vitro experiment was done to demonstrate one effect of light therapy on neurons, namely the effect on ATP production. Normal Human Neural Progenitor (NHNP) cells were obtained cryopreserved through Clonetics (Baltimore, Md.), catalog # CC-2599. NHNP cells were thawed and cultured on polyethyleneimine (PEI) with reagents provided with the cells, following the manufacturers instructions. The cells were plated into 96 well plates (black plastic with clear bottoms, Becton Dickinson, Franklin Lakes N.J.) as spheroids and allowed to differentiate into mature neurons over a period of two weeks.

A Photo Dosing Assembly (PDA) was used to provide precisely metered doses of laser light to the NHNP cells in the 96 well plate. The PDA comprised a Nikon Diaphot inverted microscope (Nikon, Melville, N.Y.) with a LUDL motorized x,y,z stage (Ludl Electronic Products, Hawthorne, N.Y.). An 808 nm laser was routed into the rear epifluorescent port on the microscope using a custom designed adapter and a fiber optic cable. Diffusing lenses were mounted in the path of the beam to create a “speckled” pattern, which was intended to mimic in vivo conditions after a laser beam passed through human skin. The beam diverged to a 25 mm diameter circle when it reached the bottom of the 96 well plate. This dimension was chosen so that a cluster of four adjacent wells could be lased at the same time. Cells were plated in a pattern such that a total of 12 clusters could be lased per 96 well plate. Stage positioning was controlled by a Silicon Graphics workstation and laser timing was performed by hand using a digital timer. The measured power density passing through the plate for the NHNP cells was 50 mW/cm².

Two independent assays were used to measure the effects of 808 nm laser light on the NHNP cells. The first was the CellTiter-Glo Luminescent Cell Viability Assay (Promega, Madison, Wis.). This assay generates a “glow-type” luminescent signal produced by a luciferase reaction with cellular ATP. The CellTiter-Glo reagent is added in an amount equal to the volume of media in the well and results in cell lysis followed by a sustained luminescent reaction that was measured using a Reporter luminometer (Turner Biosystems, Sunnyvale, Calif.). Amounts of ATP present in the NHNP cells were quantified in Relative Luminescent Units (RLUs) by the luminometer.

The second assay used was the alamarBlue assay (Biosource, Camarillo, Calif.). The internal environment of a proliferating cell is more reduced than that of a non-proliferating cell. Specifically, the ratios of NADPH/NADP, FADH/FAD, FMNH/FMN and NADH/NAD, increase during proliferation. Laser irradiation is also thought to have an effect on these ratios. Compounds such as alamarBlue are reduced by these metabolic intermediates and can be used to monitor cellular states. The oxidization of alamarBlue is accompanied by a measurable shift in color. In its unoxidized state, alamarBlue appears blue; when oxidized, the color changes to red. To quantify this shift, a 340PC microplate reading spectrophotometer (Molecular Devices, Sunnyvale, Calif.) was used to measure the absorbance of a well containing NHNP cells, media and alamarBlue diluted 10% v/v. The absorbance of each well was measured at 570 nm and 600 nm and the percent reduction of alamarBlue was calculated using an equation provided by the manufacturer.

The two metrics described above, (RLUs and % Reduction) were then used to compare NHNP culture wells that had been lased with 50 mW/cm² at a wavelength of 808 nm. For the CellTiter-Glo assay, 20 wells were lased for 1 second and compared to an unlased control group of 20 wells. The CellTiter-Glo reagent was added 10 min after lasing completed and the plate was read after the cells had lysed and the luciferase reaction had stabilized. The average RLUs measured for the control wells was 3808+/−3394 while the laser group showed a two fold increase in ATP content to 7513+/−6109. The standard deviations were somewhat high due to the relatively small number of NHNP cells in the wells (approximately 100 per well from visual observation), but a student's unpaired t-test was performed on the data with a resulting p-value of 0.02 indicating that the twofold change is statistically significant.

The alamarBlue assay was performed with a higher cell density and a lasing time of 5 seconds. The plating density (calculated to be between 7,500-26,000 cells per well based on the certificate of analysis provided by the manufacturer) was difficult to determine since some of the cells had remained in the spheroids and had not completely differentiated. Wells from the same plate can still be compared though, since plating conditions were identical. alamarBlue was added immediately after lasing and the absorbance was measured 9.5 hours later. The average measured values for percent reduction were 22%+/−7.3% for the 8 lased wells and 12.4%+/−5.9% for the 3 unlased control wells (p-value=0.076). These alamarBlue results support the earlier findings in that they show a similar positive effect of the laser treatment on the cells.

Increases in cellular ATP concentration and a more reduced state within the cell are both related to cellular metabolism and are considered to be indications that the cell is viable and healthy. These results are novel and significant in that they show the positive effects of laser irradiation on cellular metabolism in in-vitro neuronal cell cultures.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

What is claimed is:

1. A method for the treatment of stroke in a subject in need of such treatment, said method comprising delivering a neuroprotective effective amount of light energy having a wavelength in the visible to near-infrared wavelength range to a target area of the brain of the subject that includes an area of infarct and surrounding tissue, wherein delivering the neuroprotective effective amount of light energy comprises delivering a predetermined power density of light energy to the target area of the brain, wherein the predetermined power density is a power density of at least about 0.01 mW/cm² at the target area.

2. A method according to claim 1 wherein the predetermined power density is a power density selected from the range of about 0.01 mW/cm² to about 100 mW/cm².

3. A method according to claim 2 wherein the predetermined power density is selected from the range of about 0.01 mW/cm² to about 15 mW/cm².

4. A method according to claim 1 wherein the light energy has a wavelength of about 630 nm to about 904 nm.

5. A method according to claim 4 wherein the light energy has a wavelength of about 780 nm to about 840 nm.

6. A method according to claim 1 wherein delivering a neuroprotective effective amount of light energy to the target area of the brain comprises placing a light source in contact with a region of skin adjacent the target area of the brain.

7. A method according to claim 1 wherein delivering a neuroprotective effective amount of light energy to the target area of the brain comprises placing a light source in contact with a region of skin contralateral the target area of the brain.

8. A method according to claim 1 wherein delivering a neuroprotective effective amount of light energy to the target area of the brain of the subject comprises determining a surface power density of the light energy sufficient to deliver the predetermined power density of light energy to the target area of the brain.

9. A method according to claim 8 wherein determining a surface power density of the light energy sufficient to deliver the predetermine power density of light energy to the area of the brain comprises determining the surface power density of the light energy sufficient for the light energy to traverse the distance between the skin surface and the area of the brain.

10. A method according to claim 9 wherein determining the surface power density further comprises determining the surface power density sufficient to penetrate the skull.

11. A method according to claim 1, wherein the method begins at least about 6 hours following the stroke.

12. A method according to claim 1, wherein the treatment proceeds for a period of about 30 seconds to about 2 hours.

13. A method of increasing the production of ATP by neurons, comprising:
irradiating neurons with light energy having a wavelength in the near infrared to visible portion of the electromagnetic spectrum for at least about 1 second;
wherein the power density of said light energy at the neurons is at least about 0.01 mW/cm².

14. A method according to claim 13 wherein the predetermined power density is a power density selected from the range of about 0.01 mW/cm² to about 100 mW/cm².

15. A method according to claim 14 wherein the predetermined power density is less than about 15 mW/cm².

16. A method according to claim 13 wherein the light energy has a wavelength of about 630 nm to about 904 nm.

17. A method according to claim 16 wherein the light energy has a wavelength of about 780 nm to about 840 nm.
